(19) United States
(12) Patent Application Publication
(10) Pub. No.: US 2020/0383775 A1
(43) Pub. Date: Dec. 10, 2020

(54) INTRAOCULAR LENS AND METHODS FOR OPTIMIZATION OF DEPTH OF FOCUS AND THE IMAGE QUALITY IN THE PERIPHERY OF THE VISUAL FIELD

(71) Applicant: VOPTICA, S.L, Murcia (ES)

(72) Inventors: Pablo ARTAL SORIANO, Murcia (ES); Harilaos GINIS, Murcia (ES); Maria BARRERA VERDEJO, Murcia (ES); Eloy VILLEGAS RUIZ, Murcia (ES); Pedro PRIETO CORRALES, Murcia (ES); Juan TABERNERO DE PAZ, Murcia (ES)

(73) Assignee: VOPTICA, S.L, Murcia (ES)

(21) Appl. No.: 16/508,807

(22) Filed: Jul. 11, 2019

(30) Foreign Application Priority Data
Jun. 7, 2019 (ES) 201930521

(51) Int. Cl. A61F 2/16

(52) U.S. Cl. CPC A61F 2/164; A61F 2002/1681; A61F 2230/0008; A61F 2230/0006; A61F 2230/0013

(57) ABSTRACT

Intraocular tens and methods for optimization of depth of focus and the image quality in the periphery of the visual field. The intraocular lens (600) comprises a central part and a peripheral part, the central part being the optical part (600) and the peripheral part comprising mechanical fasteners (603), and the central part comprises: an aspherical concave anterior surface (601), which is the surface closest to the iris of the eye once the lens (600) has been implanted in the eye, and an aspherical convex posterior surface (602), which is the surface closest to the retina of the eye once the lens (600) has been implanted in the eye, such that the radius of curvature of the posterior surface (602) of the central part is smaller than the radius of curvature of the anterior surface (601) of the central part, with a ratio between radii of between 2 and 6, and the mechanical fasteners (603) are arranged at an angle (605) of between 0° and 10° with respect to a plane passing through the joints between the central part a the peripheral part and which is perpendicular to the optical axis of the eye in which the lens (600) is intended to be implanted.

### FIELD OF INVENTION

The present invention refers to an intraocular lens used in ophthalmology, specifically for cataract surgery. Additionally, it refers to methods for optimization of depth of focus through customized asphericity and the image quality in the periphery of the visual field that use said intraocular lenses.

### BACKGROUND OF THE INVENTION

The crystalline lens is a complex structure, whose function has been a topic of study for centuries. It presents a gradient in its refractive index ranging from about 1.390 at the lens surface to about 1.409 at its core. This gradient is achieved thanks to a variable concentration of proteins in the cytoplasm of lens fiber cells. Despite the high presence of proteins, the healthy crystalline lens shows a high transparency due to the compact distribution of proteins. In addition, the crystalline lens has aspherical surfaces, which reduces spherical aberration in the eye. On the other hand, the design of the human eye also allows the optimization of the formation of the image at the periphery in terms of aberrations and field curvature with respect to the shape of the retina.

Cataracts are a pathological condition in which the natural crystalline lens has lost its transparency and the formation of image in the retina is deteriorated mainly due to the scattering of light. In most cases, to solve this problem, cataract surgery is performed by which the damaged lens is removed from the eye and a specific implant (an intraocular lens 100 is placed to substitute the extracted crystalline lens.

Intraocular lenses have been used in ophthalmology for more than half a century. Since then, IOL implantation has become the most common and successful surgery, not only in the field of ophthalmology, but in the field of medicine in general. The implant is chosen to provide the refractive power needed to optimize the focal point in the fovea, the highest resolution area of the retina, thus replacing the patient's crystalline lens. The central visual field of the pseudo-phakic eye (with the lens replaced by an IOL) is characterized by good image quality, almost entirely limited by the post-operative sphero-cylindrical error, or even in some cases and depending on the type of implant used, by the induction of spherical aberration.

In most cases, the optical quality at the periphery of the visual field is not considered to be one of the most important points in the design of an IOL, since, for major eccentricities in the retina, the number of photoreceptor cells decreases and neuronal convergence increases. Due to these physiological limitations of the retina, IOL manufacturers have designed ocular implants focusing solely on image quality in the central area of the fovea, regardless of what occurs in the far periphery of the visual field. While the natural crystalline lens has properties adapted to the shape of the retina, IOLs do not contemplate this phenomenon at all. However, there are specific applications where the quality of peripheral vision can have a great impact on the quality of vision, such as detecting the direction of movement of visual patterns or even simpler activities such as detecting small peripheral objects. It is possible that the mismatch between the field curvature provided by conventional IOLs and the shape of the retina produces very low contrast and quality images that are related to accidents due to the non-detection of obstacles in various areas of the visual field.

Another common phenomenon that occurs in the distant periphery in pseudo-phakic patients, of a rather unclear etiology, is the presence of dark shadows, and is referred to as negative dysphotopsy.

Although the peripheral behaviour of pseudophakic eyes has not yet been studied in depth, there is some evidence that IOLs are not capable of reproducing the peripheral image quality that the natural crystalline lens does bring to human vision (see B. Jaeken, S. Mirabet, J. M. Marin, P. Artal, “Comparison of the Optical Image Quality in the Periphery of Phakic and Pseudophakic Eyes”, Invest Ophthalmol Vis Sci, 54, 3594-3599, (2013)). Intraocular lenses are specifically designed to facilitate surgical techniques (such as ease of implantation), with the goal of regaining emmetropia and prioritizing central vision.

In most cases, current intraocular lenses induce problems such as increased astigmatism and other aberrations on the periphery of the pseudophakic eye's visual field. Although in principle these phenomena may be imperceptible directly by the patient (as would be the presence of blur in the central vision), there is evidence that they play a role in the patient's spatial orientation and safety.

On the other hand, there are currently several IOLs with different asphericity values, both to compensate for corneal asphericity and to extend the depth of focus and allow acceptable vision at various distances. A problem with these IOLs is that they provide a unique asphericity value. However, each patient has a cornea with a different value of spherical aberration to be considered. In addition, each patient has a different sensitivity to the final net amount of spherical aberration after implantation of the IOL.

On the other hand, objective and visual measurements on each patient can be made using adaptive optics visual simulation technology (see P. A. Piers, E. J. Fernandez, S. Manzanera, S. Norrby, P. Artal, “Adaptive optics simulation of intraocular lenses with modified spherical aberration” Invest. Ophthalmol. Vis. Sci. 45, 4601-4610 (2004)).

Many types of IOLs have already been implemented in the patent and scientific literature. For example, U.S. Pat. No. 4,504,982 A (Burk) describes a generic aspherical lens for intraocular implantation that has a biconvex shape. However, in its design there is no provision for peripheral vision.

CA2587432C (Norrby) describes a method for selecting an intraocular lens, involving only optical and anatomical measurements that are subsequently used to calculate the power and spherical aberration of the required IOL. This invention does not consider possible arrangements for peripheral image quality and, furthermore, does not include the preoperative subjective (psychophysical) evaluation of patients.

AU2007247491B2 (Gerlach) describes a method for designing an intraocular lens with a given asphericity to compensate for corneal aberrations, considering the possible eccentricity of the pupil. The same principles are described in a later document by the same inventors (U.S. Pat. No. 8,235,525 B2 (Lesage)). The above mentioned invention does not include provisions for peripheral image quality.

Document WO2005098518A1 (Piers) describes a hybrid lens with at least one diffractive element that is used to control the total longitudinal chromatic aberration of the eye.

CA2431470C (Piers) presents an IOL design that compensates for the spherical aberration of the cornea without mentioning its customization.

U.S. Pat. No. 10,010,407 B2 (Rosen), describes an IOL design to improve wavefront specific aberrations such as coma or astigmatism for the peripheral visual field for angles up to 40 degrees, without reference to peripheral image quality and distortion.

There is currently a need in the intraocular lens market for an implant that provides an optimization of field curvature to fit it to the shape of the retina to optimize image quality in the far periphery of the visual field. In addition, it is desirable that the lenses have a range of spherical net aberration values to be customized to increase the depth of field customized to each patient.

### SUMMARY OF THE INVENTION

The object of the present invention is to provide an intraocular lens that solves the aforementioned inconveniences.

The invention provides an intraocular lens comprising a central part and a peripheral part, the central part being the optical part and the peripheral part comprising mechanical fasteners (haptics), in which the central part comprises:
an aspherical concave anterior surface, which is the surface closest to the iris of the eye once the lens has been implanted in the eye, and
an aspherical convex posterior surface, which is the surface closest to the retina of the eye once the lens has been implanted in the eye, such that:
the radius of curvature of the posterior surface of the central part is smaller than the radius of curvature of the anterior surface of the central part, with a ratio between radii of between 2 and 6, and
the mechanical fasteners are arranged at an angle of between 0° and 10° with respect to a plane passing through the joints between the central part and the peripheral part and which is perpendicular to the optical axis of the eye in which the lens is intended to be implanted.

This intraocular lens, when implanted in the posterior chamber of the eye, simulates the natural imaging formation properties of the crystalline lens, especially in the far periphery of the visual field, optimizing the field curvature of the implanted eye to adapt it appropriately to the shape of the retina.

This intraocular lens results in an improved image formation in terms of sharpness, peripheral refraction and geometric distortion.

The invention also provides methods for optimizing depth of focus and image quality in the periphery of the visual field using said intraocular lens, according to claims 8 and 9.

Other characteristics and advantages of the present invention will be derived from the detailed description that follows from an illustrative embodiment of its object in relation to the accompanying figures.

### BRIEF DESCRIPTION OF THE DRAWINGS

The object of the present invention will be illustrated below in a non-limiting way, making reference to the drawings that are accompanied, in which:

FIG. 1 shows a section of a human eye and the focusing of objects in the center of the visual field and on the periphery of the visual field.

FIG. 2 shows a section of a human eye with a prior art intraocular lens implanted in the posterior chamber of the eye, and the focusing of objects in the center of the visual field and on the periphery of the visual field.

FIG. 3 shows a section of a human eye with an intraocular lens of the invention implanted in the posterior chamber of the eye, and the focusing of objects in the center of the visual field and on the periphery of the visual field.

FIG. 4A shows one embodiment of an intraocular lens according to the invention.

FIG. 4B shows another embodiment of an intraocular lens according to the invention.

FIG. 5 schematically shows a visual simulator of adaptive optics and its use in the method to optimize depth of focus and image quality at the periphery of the visual field of invention.

### DETAILED DESCRIPTION OF THE INVENTION

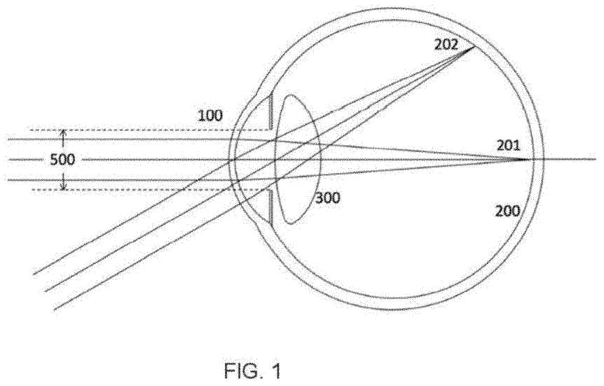

FIG. 1 shows a section of the human eye. The light 500 entering through the pupil is focused by the cornea 100 and the lens of the crystalline 300 on the retina 200. Objects in the center of the visual field are focused on the central part 201 of the retina, while objects on the periphery of the visual field are focused on a peripheral point 202 of the retina.

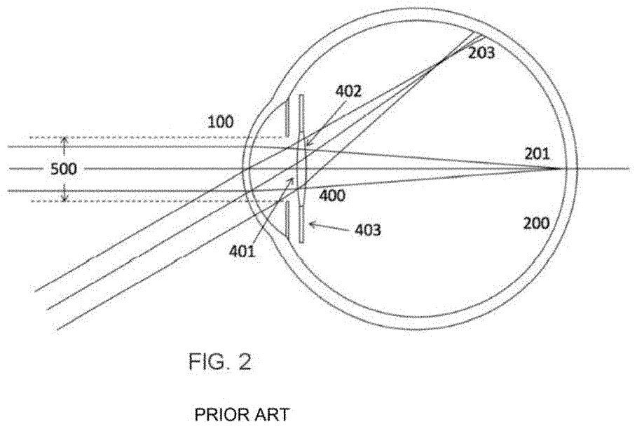

FIG. 2 shows an eye with a prior art intraocular lens 400 implanted in the posterior chamber of the eye. The light 500 entering through the pupil is focused by the cornea 100 on the retina 200. The intraocular lens 400 has an anterior surface 401, a posterior surface 402 and mechanical fasteners or haptics 403. Objects in the center of the visual field are focused on the central part 201 of the retina, while objects on the periphery of the visual field are focused on a peripheral point 203 of the retina. This peripheral point 203 is generally different from the peripheral focus point 202 prior to cataract surgery. In addition, rays entering the eye from the periphery of the visual field are strongly aberrated and the image at peripheral point 203 is out of focus.

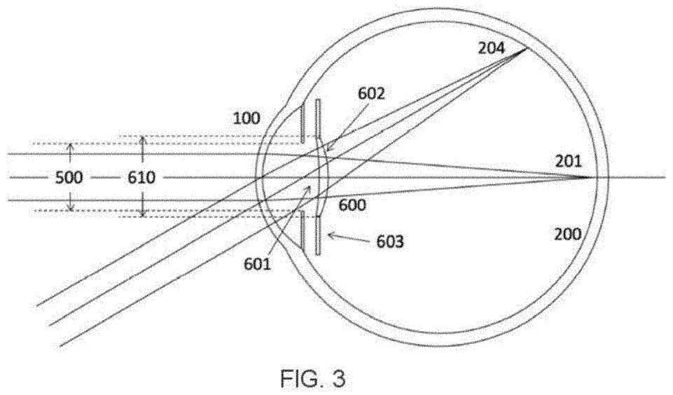

FIG. 3 shows an intraocular lens 600 of the invention implanted in the posterior chamber of the eye. An eye is depicted with an improved intraocular lens 600, implanted in the posterior chamber. The light 500 entering through the pupil is focused by the cornea 100 on the retina 200. The intraocular lens 600 has an anterior surface 601, a posterior surface 602, an optical zone 610, and mechanical fasteners (haptics) 603. Objects in the center of the visual field are focused on the central part 201 of the retina, while objects in the periphery of the visual field are focused on a peripheral point 204 of the retina. This peripheral point 204 is close to the peripheral focus point 202 prior to cataract surgery. In addition, rays entering the eye from the periphery of the visual field are less aberrated and the image at peripheral point 204 is more focused.

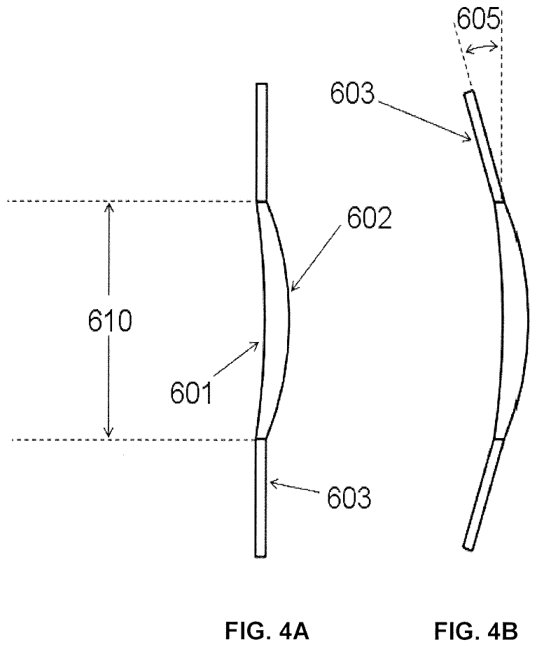

FIGS. 4A and 4B show two embodiments of intraocular lenses of the invention. FIG. 4A shows the intraocular lens 600, with an anterior zone 601 that is concave and a posterior zone 602 that is convex, its optical zone 610, about 6 mm in diameter, and the mechanical fasteners (haptics) 603. FIG. 4B shows the mechanical fasteners (haptics) 604 with a certain angle 605 to a plane passing through the junctions between the central part and the peripheral part. This plane shall be perpendicular to the optical axis of the eye in which the lens is intended to be implanted.

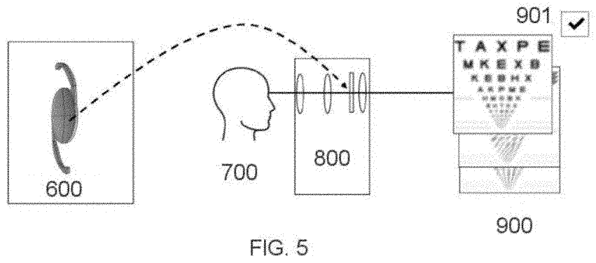

FIG. 5 shows an adaptive optics visual simulator 800, which simulates different profiles of intraocular lenses 600 for a patient 700. The patient 700 evaluates the results of the simulations 900 through a visual test at different distances (i.e. far, intermediate, close) and chooses the most optimal result 901 according to his/her visual necessities. This is used to determine the net value of asphericity on the IOL to be implanted.

The present invention refers to the design of a new posterior chamber intraocular lens 600 that simulates the image formation properties of the natural crystalline lens, in the specific referring to the induced field curvature and to the improvement of the visual quality on the far periphery of the retina. Moreover, it can offer extended depth of focus both for central and peripheral vision and therefore reduce the dependence on spectacles postoperatively as well as allow for variability of the shape of peripheral retina. The IOL 600 is manufactured from one of the existing materials suitable of intraocular implantation such as hydrophobic acrylic, hydrophilic acrylic, silicone and can be produced either by machining or by molding.

The intraocular lens 600 has a central part with a diameter between 5 and 7 mm (preferably around 6 mm), with the required optical properties, and a peripheral part that extends to a diameter between approximately 10 and 14 mm (the haptics or mechanical fasteners 603) that help center and stabilize the intraocular lens 600 in the lens capsule once the cataract crystalline lens has been removed. The haptics 603 are selected from a range of designs established in the scientific literature. In the surgical process, the lens 600 is bent and injected into the prepared capsule through a small incision in the limbus or peripheral cornea.

In an embodiment of the intraocular lens 600, it is manufactured by molding acrylic hydrophobic material with a refractive index of approximately 1.53. The anterior surface 601 of the intraocular lens 600 is concave aspherical, and the posterior surface 602 is convex aspherical. The optical part 610 of the lens 600 is circular and has a diameter of approximately 6 mm. The anterior surface 601 has a negative power when submerged in the aqueous humour of the eye (or a similar aqueous solution, such as saline solution), while the posterior surface 602 has a positive power. The combination of these two surfaces produces a refractive power of the order of 20 D when the intraocular lens 600 is immersed in the aqueous humour of the eye (or a similar aqueous solution, such as saline). The radius of curvature of the anterior surface 601 can be changed to produce intraocular lenses 600 of the same type, but with different diopter powers, as required in cataract surgery. The posterior surface 602 has an appropriate conical constant such that the spherical aberration of the lens 600 compensates for the spherical aberration of the average cornea of a human eye. In this embodiment, the total spherical aberration of the eye is practically zero, achieving optimal image quality for central and peripheral vision. The haptics 603 form an angle 605 (see FIG. 4b) with respect to a plane passing through the junctions between the central part and the peripheral part and which is perpendicular to the optical axis of the eye in which the lens 600 of approximately 2 degrees is intended to be implanted.

In another embodiment of the present invention, the intraocular lens 600 is manufactured by machining or molding hydrophobic acrylic material with a refractive index of between about 1.46 to about 1.55. The anterior surface 601 of the intraocular lens 600 is concave aspherical and the posterior surface 602 is convex aspherical. The optical part 610 of the lens 600 is circular and has a diameter of approximately 5 to 7 mm. The anterior surface 601 has a negative power when submerged in the aqueous humour of the eye (or a similar aqueous solution, such as saline solution), while the posterior surface 602 has a positive power. The combination of these two surfaces produces a refractive power of the order of 20 D when the intraocular lens 600 is immersed in the aqueous humour of the eye (or a similar aqueous solution, such as saline). The radius of curvature of the anterior surface 601 can be changed to produce intraocular lenses 600 of the same type, but with different diopter powers, as required in cataract surgery. The posterior surface 602 has an appropriate conical constant such that the total power of the intraocular lens 600 remains constant, but that the negative spherical aberration of the lens 600 induces a concrete value equal to or greater (in absolute value) than that of the average cornea of a human eye. In this embodiment, the total spherical aberration of the eye can be adjusted between values close to zero and a negative value approximately 5 times greater (in magnitude) than that of the average cornea of a human eye. In this way, a greater compromise between image quality and depth of focus can be achieved. The optical part 610 has a diameter of approximately 6 mm and the haptics 603 form an angle 605 with respect to a plane that passes through the joints between the central part and the peripheral part and that is perpendicular to the optical axis of the eye in which the lens 600 is to be implanted, which varies between 0 and approximately 10°.

In another embodiment, the intraocular lens 600 is manufactured by machining or molding any material in the hydrophobic acrylic, hydrophilic acrylic, or silicone group that has refractive indices typically between approximately 1.46 and approximately 1.55. The anterior surface 601 of the intraocular lens 600 is concave aspherical and the posterior surface 602 is aspherical convex. The optical part 610 of the lens 600 is circular and has a diameter ranging from approximately 5 to 7 mm. The anterior surface 601 has a negative power when submerged in the aqueous humour of the eye (or a similar aqueous solution, such as saline solution), while the posterior surface 602 has a positive power. The combination of these two surfaces produces a refractive power of the order of 20 D when the intraocular lens 600 is immersed in the aqueous humour of the eye (or a similar aqueous solution, such as saline). The radius of curvature of the anterior surface 601 can be changed to produce intraocular lenses 600 of the same type, but with different diopter powers, as required in cataract surgery. The balance of diopter power between the anterior 601 and posterior 602 surfaces can be changed to compensate for total or partial peripheral astigmatism in the eye. Both surfaces, anterior 601 and posterior 602, may have conical constants defined in such a way that the power of the intraocular lens 600 remains almost constant but the negative spherical aberration of the lens 600 has a particular value equal to or greater (in absolute value) than that of the average human cornea. In this embodiment, the total spherical aberration in the eye can be adjusted from practically zero to a value approximately 5 times greater than the spherical aberration value of the normal eye (in absolute value). In this way, the best compromise between image quality and depth of focus is achieved. The optical zone 601 has a diameter of approximately 6 mm and the haptics 603 form an angle 605 with respect to a plane passing through the joints between the central part and the peripheral part and which is perpendicular to the optical axis of the eye in which the lens 600 is to be implanted, which varies between approximately 0 and approximately 10 degrees. The angulation 605 of the 603 haptics 603 controls the axial depth at which the intraocular lens 600 is implanted into the lens capsule. This adjustment, combined with the power balance between the anterior surface 601 and the posterior surface 602 can be varied to control the shape of the field curvature in the eye and make it match the shape of the retina. This adjustment helps correct peripheral refractive errors in the eye, a task that may be of particular importance for the control of myopia development following congenital cataract surgery. In a similar embodiment, the optical part 610 of the lens 600 can be oval. The longest axis of the oval would be implanted horizontally to reduce the phenomena associated with light diffusion and glare produced by the edge of the lens 600 in the distant temporal field of vision.

In another embodiment, the lens 600 is manufactured with one of the materials mentioned above, the anterior surface 601 of the lens 600 being concave aspherical and the posterior surface 602 being convex aspherical. The optical part 610 of the lens 600 is circular and has a diameter between approximately 5 and 7 mm. The central and peripheral refraction of a patient 700 is measured, as well as the topography of his cornea and the central and peripheral biometries. Corneal topography information, the axial placement provided for the intraocular lens 600 during implantation, as well as central and peripheral biometries are used to select from a range the angulation 605 of the haptics 603 and the balance between the anterior 601 and posterior 602 surface curvature radii to induce a desired central and peripheral post operative refraction.

In another embodiment, the lens 600 is manufactured with one of the materials mentioned above, the anterior surface 601 of the lens 600 being concave aspherical and the posterior surface 602 being convex aspherical. The optical part 610 of the lens 600 is circular and has a diameter of between approximately 5 mm to approximately 7 mm. The vision of patient 700 can be evaluated thanks to an adaptive optics simulator 800, in which a specific amount of spherical aberration (typically negative) is determined to optimize each patient individually. The optimization aims at maximizing the depth of focus without significantly sacrificing the quality of distant vision. When this value is determined, the patient's corneal topography and biometry are used to select from a range of available intraocular lenses 600 that have different conical constants on at least one of the surfaces 601 or 602, to provide adequate post-operative refraction and depth of field.

A method for optimizing depth of focus and the image quality in the periphery of the visual field uses intraocular lenses 600 of those described above and comprises the following steps (FIG. 5):
to provide a series of intraocular lenses 600 of different net asphericity value,
to place a patient 700 in front of a visual simulator 800 with adaptive optics, which emulates different intraocular lens profiles with different net asphericity value,
realization of different simulations 900 with different intraocular lens profiles through a visual test at different distances,
selection of the optimal result 901 of the visual test, and determination of the net asphericity value of the intraocular lens, and
choice of the intraocular lens 600 presenting the determined net asphericity value, for implantation in the eye of patient 700.

Another method for optimizing depth of focus and image quality at the periphery of the visual field uses intraocular lenses 600 of those described above and comprises the following steps:
measurement of eye shape and/or refraction at the periphery, and
depending on the measurements made, to customize the shape of the intraocular lens 600 and its net asphericity, for its implantation in the eye of patient 700.

Although some embodiments of the invention have been described and represented, it is clear that modifications can be made to them within its scope, which should not be considered to be limited to those embodiments, but only to the content of the following claims.

1. Intraocular lens (600), comprising a central part and a peripheral part, the central part being the optical part (600) and the peripheral part comprising mechanical fasteners (603), characterized by the fact that the central part comprises:
an aspherical concave anterior surface (601), which is the surface closest to the iris of the eye once the lens (600) has been implanted in the eye, and
an aspherical convex posterior surface (602), which is the surface closest to the retina of the eye once the lens (600) has been implanted in the eye, such that:
the radius of curvature of the posterior surface (602) of the central part is smaller than the radius of curvature of the anterior surface (601) of the central part, with a ratio between radii of between 2 and 6, and
the mechanical fasteners (603) are arranged at an angle (605) of between 0° and 10° with respect to a plane passing through the Joints between the central part and the peripheral part and which is perpendicular to the optical axis of the eye in which the lens (600) is intended to be implanted.

2. Intraocular lens (600), according to claim 1, in which the mechanical fasteners (603) are arranged at an angle (605) of 2° with respect to a plane passing through the joints between the central part and the peripheral part and which is perpendicular to the optical axis of the eye in which the lens (600) is intended to be implanted.

3. Intraocular lens (600), according to claim 1, in which the central part has a circular aspect.

4. Intraocular lens (600), according to claim 2, in which the central part has a circular aspect.

5. Intraocular lens (600), according to claim 3, in which the central part has a diameter of between 5 and 7 mm.

6. Intraocular lens (600), according to claim 4, in which the central part has a diameter of between 5 and 7 mm.

7. Intraocular lens (600), according to claim 1, in which the central part has an oval aspect.

8. Intraocular lens (600), according to claim 2, in which the central part has an oval aspect.

9. Intraocular lens (600), according to claim 8, in which the long axis of the oval is arranged horizontally once the lens (600) has been implanted in the eye.

10. Intraocular lens (600), according to claim 1, which is made of a material selected from the group of hydrophobic acrylics, hydrophilic acrylics or silicones.

11. Intraocular lens (600), according to claim 2, which is made of a material selected from the group of hydrophobic acrylics, hydrophilic acrylics or silicones.

12. Intraocular lens (600), according to claim 3, which is made of a material selected from the group of hydrophobic acrylics, hydrophilic acrylics or silicones.

13. Intraocular lens (600), according to claim 7, which is made of a material selected from the group of hydrophobic acrylics, hydrophilic acrylics or silicones.

14. Method to optimize depth of focus and the quality of the image in the periphery of the visual field, which uses intraocular lenses (600) of claim 1, which comprises the following steps:
to provide a series of intraocular lenses (600) of different net asphericity value.
to place a patient (700) in front of a visual simulator (800) of adaptive optics, which emulates different intraocular lens profiles with different net asphericity value,
realization of different simulations (900) with different intraocular lens profiles through a visual test at different distances,
selection of the optimal result (901) of the visual test, and determination of the net asphericity value of the intraocular lens, and
choice of the intraocular lens (600) presenting the determined net asphericity value for its implantation in the patient's eye (700).

15. Method to optimize depth of focus and the quality of the image in the periphery of the visual field according to claim 14 wherein said intraocular lenses are ones in which the mechanical fasteners (603) are arranged at an angle (605) of 2° with respect to a plane passing through the joints between the central part and the peripheral part and which is perpendicular to the optical axis of the eye in which the lens (600) is intended to be implanted.

16. Method to optimize depth of focus and the quality of the image in the periphery of the visual field according to claim 14 in which the intraocular lenses are ones in which the central part has a circular aspect.

17. Method to optimize depth of focus and the quality of the image in the periphery of the visual field according to claim 14 in which the intraocular lenses are ones in which the central part has an oval aspect.

18. Method to optimize depth of focus and the quality of the image in the periphery of the visual field, which uses intraocular lenses (600) of claim 1, which comprises the following steps:
measurement of eye shape and/or refraction at the periphery, and
depending on the measurements made, to customise the shape of the intraocular lens (600) and its net asphericity for its implantation in the patient's eye (700).

19. Method to optimize depth of focus and the quality of the image in the periphery of the visual field, according to claim 18 wherein said intraocular lenses are ones in which the mechanical fasteners (603) are arranged at an angle (605) of 2° with respect to a plane passing through the joints between the central part and the peripheral part and which is perpendicular to the optical axis of the eye in which the lens (600) is intended to be implanted.

20. Method to optimize depth of focus and the quality of the image in the periphery of the visual field according to claim 18 in which the intraocular lenses are ones in which the central part has a circular or oval aspect.
